(19) United States
(12) Patent Application Publication
(10) Pub. No.: US 2007/0238147 A1
(43) Pub. Date: Oct. 11, 2007

(54) Method of detecting foreign matter

(75) Inventors: Takashi Okamoto, Hachioji-shi (JP); Yuji Okita, Hachioji-shi (JP); Satoru Chiba, Hachioji-shi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: NIPPON SUISAN KAISHA, LTD., (JP)

(21) Appl. No.: 11/727,202

(22) Filed: Mar. 23, 2007

(30) Foreign Application Priority Data
Mar. 24, 2006 (JP) 2006-082753

(51) Int. Cl. C12Q 1/04

(52) U.S. Cl. 435/34

(57) ABSTRACT

A method for detecting foreign matter such as a parasite, in particular, to provide a method for detecting parasitic on meat. A method for detecting foreign matter includes detecting fluorescence excited by irradiating the foreign matter such as a parasite with visible light. The method is suitable for detecting a meat parasite. A method for detecting a parasite includes selectively detecting fluorescence emitted from a parasite with a filter that does not transmit excitation light but that transmits fluorescence emitted from the parasite. An apparatus for detecting a parasite includes a light source that illuminates a specimen with a visible light having a predetermined wavelength, a filter that absorbs diffuse reflection of excitation light, a camera that shoots fluorescence from the specimen, an image processing unit, and a monitor.

### TECHNICAL FIELD

The present invention relates to a method for detecting a foreign matter. In particular, the present invention relates to a method for detecting a parasite that is parasitic on fishery products, livestock meat, and the like.

### BACKGROUND ART

It is known that various parasites are present in any natural or cultured fishery product. Various methods for preventing or eliminating parasites have been designed. However, there are parasites in which no effective prevention or elimination method is found. These parasites often impair the commercial value of fishery products. In this case, the detection of parasites in products processed from fishery products is an industrially important challenge.

For example, in the production of Theragra chalcogramma fillets, a method what is called candling is employed to detect such parasites. Candling is a method of inspecting fillets for parasites with transmitted light. Specifically, candling is a method in which fillets are continuously fed on a belt conveyor that transmits light, the fillets are illuminated by a light source such as a fluorescent lamp from underneath, and an inspector examines the fillets for parasites from above. This method is an excellent method because fillets are comprehensively examined for contamination with foreign matter, such as residual skins and bones, as well as parasites, and then the contaminants found are eliminated.

When the parasite is a parasite having a certain level of size, for example, Anisakis, the parasite is relatively easily detected by candling. However, a smaller parasite increases the difficulty in detecting the parasite.

Furthermore, in the case where candling is performed in a place at which fillets are produced, from the standpoint of productivity and the maintenance of fillet freshness, the belt conveyor often needs to operate at a predetermined speed or more during candling. In this case, the difficulty in detecting small parasites is further increased.

Ichthyophonus hoferi is exemplified as a small parasite. Ichthyophonus hoferi has been known as a parasite in marine fish since antiquity. In recent years, it has been found that Ichthyophonus hoferi is parasitic on Theragra chalcogramma (Non-Patent Document 1). The report reveals that in muscles (edible part) of Theragra chalcogramma parasitized, a certain type of protease activity is increased, thereby disadvantageously softening the flesh of a product from Theragra chalcogramma. However, this parasite is much smaller than Anisakis. Thus, it is very difficult to detect the parasite by candling.

As a method other than candling, Patent Document 1 discloses a method for detecting a parasite by irradiating the parasite with ultraviolet rays. The specification describes that “when a parasite or a parasite egg in a fillet is irradiated with ultraviolet wavelength light, the parasite or parasite egg is excited by absorbing ultraviolet rays to emit blue light within the visible light wavelength range. This phenomenon is not observed in the case of the illumination with a known illuminator”.

In many types of fish, it is known that muscles of fishery products are subjected to ultraviolet-ray irradiation to emit fluorescence (Non-Patent Document 2).

[Patent Document 1] Japanese Unexamined Patent Application Publication No. 1-311253

[Non-Patent Document 1] Kimura et al, Fish. Sci. 68(Suppl.): 1549-1552, 2002

[Non-Patent Document 2] Harry, J. Sci. Food Agric. 33: 1135-1142, 1982

### DISCLOSURE OF THE INVENTION

### Problems to be Solved by the Invention

Accordingly, the object of the present invention is to provide a method for detecting a parasite. In particular, the object of the present invention is to provide a method for detecting a parasite that is parasitic on meat.

### MEANS FOR SOLVING THE PROBLEMS

To overcome the above-described problems, the inventors have conducted intensive studies and found that a parasite is subjected to visible-light irradiation to fluoresce, but fish does not fluoresce regardless of freshness. This finding resulted in completion of the present invention.

A method according to the invention for detecting a parasite is summarized in items (1) to (7):

(1) A method for detecting a parasite including detecting fluorescence excited by irradiating the parasite with visible light.

(2) The method for detecting a parasite described in item (1), wherein the parasite is parasitic on meat.

(3) The method for detecting a parasite described in item (1) or (2), wherein fluorescence emitted from the parasite is selectively detected by using a filter that does not transmit excitation light but that transmits fluorescence emitted from the parasite.

(4) The method for detecting a parasite described in item (1), (2), or (3), wherein the parasite is a parasite that fluoresces in response to being subjected to visible-light irradiation.

(5) The method for detecting a parasite described in item (4), wherein the parasite is Ichthyophonus hoferi.

Furthermore, a method according to the invention for detecting foreign matter is summarized in items (6) and (7):

(6) A method for detecting foreign matter including detecting fluorescence excited by irradiating meat with visible light.

(7) The method for detecting foreign matter described in item (6), wherein the foreign matter is any of wood, fibers, paper, eggshells, rubber, and insects.

[Advantages]

According to the present invention, a parasite that is parasitic on meat is efficiently detected. Furthermore, the detection of fluorescence permits analysis to be performed by image analysis without visual inspection.

### BEST MODE FOR CARRYING OUT THE INVENTION

The present invention will be described in detail. The term “meats” in the present invention refers to meats such as fishery products, beef, pork, and chicken. The present invention is applied to meats that may be parasitized by a parasite. The term “fishery products” in the present invention refers to fishery product generally eaten. The shape of a product processed is not particularly limited. If an obstacle, such as a skin or a shell, which significantly attenuates the amount of light is present between a light source and a parasite, the obstacle is preferably removed in advance. For example, in the case of a method of processing fish into fillets, unlike a method, such as candling, of using transmitted light, in the present invention, there is no problem even if each of the fillets has a skin on a side thereof. It is also known that meat of livestock and chicken other than fishery products are subjected to ultraviolet irradiation to fluoresce. The detecting method using visible-light irradiation of the present invention is more suitable than a detection method using ultraviolet-ray irradiation.

Examples of the known parasite parasitic on meat include Ichthyophonus, Anisakis, sporozoan, gnathostoma, and Paragonimus, which are parasitic on fishery products; Taenia saginata which is parasitic on beef; and Taenia solium and trichinella, which are parasitic on pork.

In the present invention, the term “visible light” refers to visible light with a wavelength of 400 to 800 nm. The wavelength of excitation light differs in response to a target parasite. For example, Ichthyophonus is irradiated with violet to green light (wavelength: 400 to 550 nm) to fluoresce. In particular, blue-light (wavelength: 430 to 490 nm) irradiation is preferred because Ichthyophonus emits strong fluorescence. The use of a light source for emitting excitation light possibly having only a predetermined wavelength range reduces noise other than fluorescence emitted from a target parasite and is thus preferred. In the case where such a light source is not easily available, the use of a combination of a common light source and a filter that transmits only a predetermined wavelength exerts an equivalent effect. An example of the filter usable is a commercially available filter that transmits a predetermined wavelength produced by Fuji Photo Film Co. Ltd.

According to the present invention, in the case where a parasite is detected by visual inspection or with a camera or the like, when fluorescence emitted from the parasite is weak, only fluorescence can surely be observed by absorbing diffuse reflection on a surface of a fishery product of excitation light with an optical filter. Any filter may be used as the optical filter as long as the optical filter absorbs the wavelength of excitation light and transmits fluorescence emitted from the parasite. A commercially available ultraviolet-absorbing filter produced by Fuji Photo Film Co. Ltd. is exemplified.

The method of the present invention is a very safe method that does not use ultraviolet rays. Thus, a parasite can be detected through visual inspection by an inspector in the same way as in candling. Furthermore, the method of the present invention is a low-noise method in which only a parasite fluoresces. Thus, the degree of parasite infestation can be automatically determined by a computer, i.e., by capturing an image with a digital camera, a CCD camera, or the like and then performing image processing, such as binarization, with a commercially-available image-analyzing apparatus combined with the camera,

A high-sensitivity camera is preferably used as the camera because fluorescence emitted from a parasite is not always strong light. For example, a 1.5-megapixel high-sensitivity camera is used. Furthermore, a high-resolution image-analyzing apparatus is used as the image-analyzing apparatus.

We demonstrated that foreign matter, such as wood, fibers, paper, eggshells, rubber, and insects, is detected by the same theory as that of the detection of the parasite described above. In the case where the foreign matter is attached to meat that does not fluoresce, the foreign matter can be detected because these materials are subjected to visible-light irradiation to fluoresce. Furthermore, other materials that fluoresce in response to being subjected to visible-light irradiation can also be detected.

Non-limiting examples of the present invention will be described below.

### EXAMPLES

### Example 1

Theragra chalcogramma caught in the Bering Sea was cut into three pieces, and then the skin was removed to obtain fresh fillets. Candling demonstrated that the fillets were parasitized by Ichthyophonus (FIG. 1). In the photograph of FIG. 1, small black spots scattered over the fillet were Ichthyophonus. It was difficult to momentarily distinguish Ichthyophonus because other foreign matter, a muscle plate, and the like were observed. Then, the fillet was irradiated with blue light by a method shown in FIG. 2 and photographed with a digital camera. FIG. 3 shows the resulting image (photograph). The fillet did not emit light, but only positions parasitized by Ichthyophonus emitted light; hence, it was momentarily and clearly identified that the fillet was parasitized.

Devices used: light source (blue LED), Denol Day Lamp (manufactured by PIAA Corporation); digital camera, Dimage F200 (manufactured by Minolta Co. Ltd., (at present, Konica Minolta Photo Imaging, Inc.)); filter transmitting particular wavelength, BPB-45 (manufactured by Fuji Photo Film Co. Ltd.); ultraviolet-absorbing filter, SC-52 (manufactured by Fuji Photo Film Co. Ltd.)

### Comparative Example 1

The same fillet used in Example 1 was irradiated with ultraviolet rays (365 nm) by a method shown in FIG. 4 and photographed with a digital camera. FIG. 5 shows the resulting image (photograph). It was significantly difficult to identify positions parasitized by Ichthyophonus because of diffuse reflection from a surface of the fillet and fluorescence emitted from the fillet subjected to ultraviolet-ray irradiation.

Devices used: light source (ultraviolet lamp), UVL-56 (manufactured by UVP, LLC); digital camera, Dimage F200 (manufactured by Minolta Co. Ltd., (at present, Konica Minolta Photo Imaging, Inc.))

### Comparative Example 2

The same fillet used in Example 1 was irradiated with ultraviolet rays (365 nm) by a method shown in FIG. 6 and photographed with a digital camera. A difference between Comparative Example 1 and Comparative Example 2 is that the same filter (ultraviolet-absorbing filter) as in Example was used in front of the lens of the digital camera. FIG. 7 shows the resulting image (photograph). Although diffuse reflection was absorbed in the filter, fluorescence emitted from the fillet was not absorbed. Unlike Example, positions parasitized by Ichthyophonus and the fillet were not clearly distinguished.

Devices used: light source (ultraviolet lamp), UVL-56 (manufactured by UVP, LLC); digital camera, Dimage F200 (manufactured by Minolta Co. Ltd., (at present, Konica Minolta Photo Imaging, Inc.)); ultraviolet-absorbing filter, SC-52 (manufactured by Fuji Photo Film Co. Ltd.)

### Example 2

A specimen (fillet of fish parasitized by Ichthyophonus) was irradiated with light from two directions at an angle of 45°, light being emitted from a blue LED through a blue bandpass filter (manufactured by FUJIFILM Corporation). The specimen was recorded with a high-sensitivity video camera through a yellow sharp-cut filter (No. 52, manufactured by FUJIFILM Corporation). The resulting image was processed by an image processing apparatus for distinguishing fluorescence emitted from the parasite and observed on a monitor.

Although bones and muscles present in the fillet emitted fluorescence, fluorescence from the parasite was distinguished by image processing. The results demonstrated that the presence or absence of parasite parasitism was distinguished by image processing.

### Example 3

A test whether foreign matter other than parasites can be detected was conducted with the same apparatus as in Example 1. Wood (toothpicks and disposable chopsticks), natural fibers (hemp lines and paper (bond paper and recycled paper), eggshells, rubber (rubber bands), insects (flies and earthworms), and plastic (rayon, polyester, and polyethylene) were subjected to the test. Among these, the plastic emitted a markedly weak fluorescence and thus was not detected with high sensitivity. However, the foreign matter except for the plastic fluoresced sufficiently and was thus detected by the method of the present invention.

### INDUSTRIAL APPLICABILITY

The presence or absence of parasite parasitism in fillets processed from meat is efficiently detected by image analysis. High-quality processed products are provided by removing meat that is parasitized by a parasite and that is not suitably processed.

### BRIEF DESCRIPTION OF THE DRAWINGS

FIG. 1 is a photograph of a fillet according to Example in candling.

FIG. 2 is an explanatory drawing of a method according to Example.

FIG. 3 is a photograph of a fillet by a method according to Example.

FIG. 4 is an explanatory drawing of a method according to Comparative Example 1.

FIG. 5 is a photograph of a fillet by a method according to Comparative Example 1.

FIG. 6 is an explanatory drawing of a method according to Comparative Example 2.

FIG. 7 is a photograph of a fillet by a method according to Comparative Example 2.

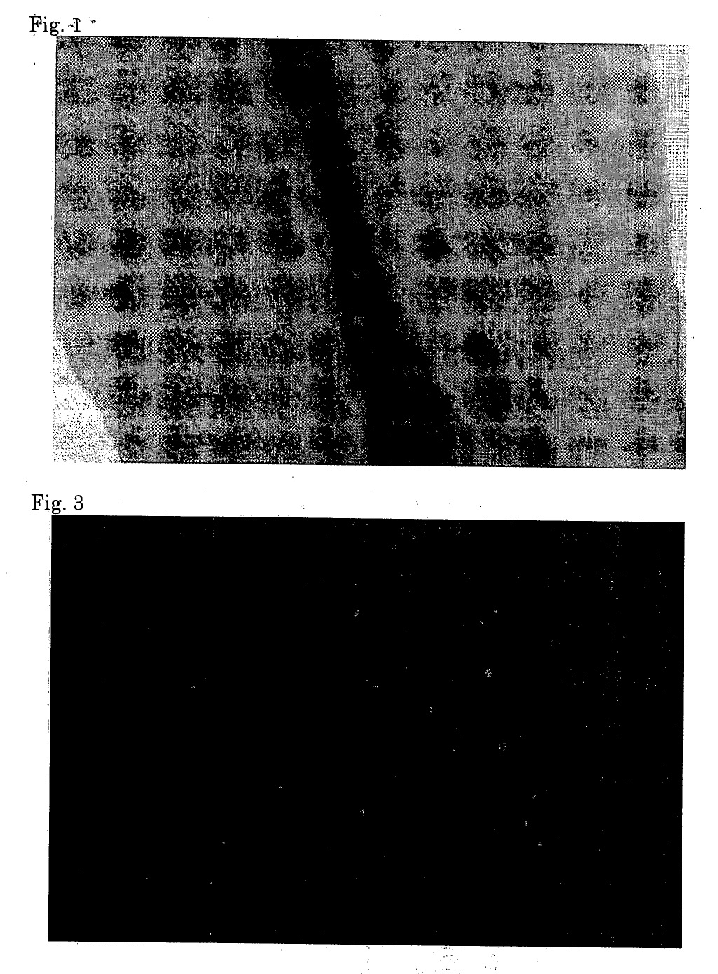

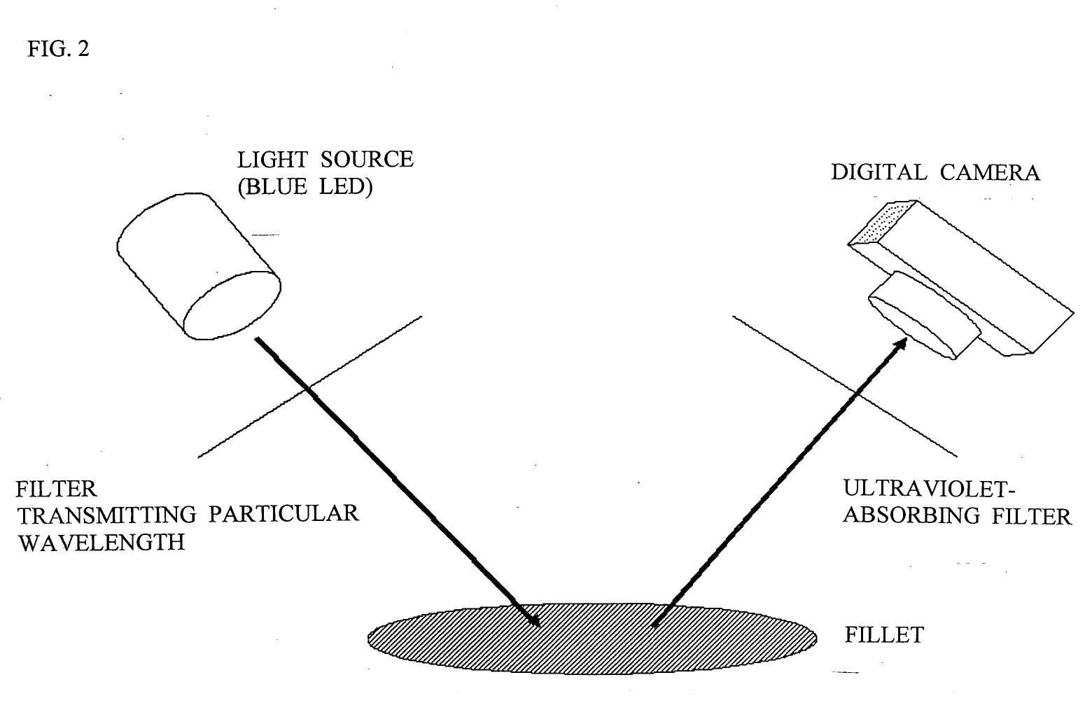

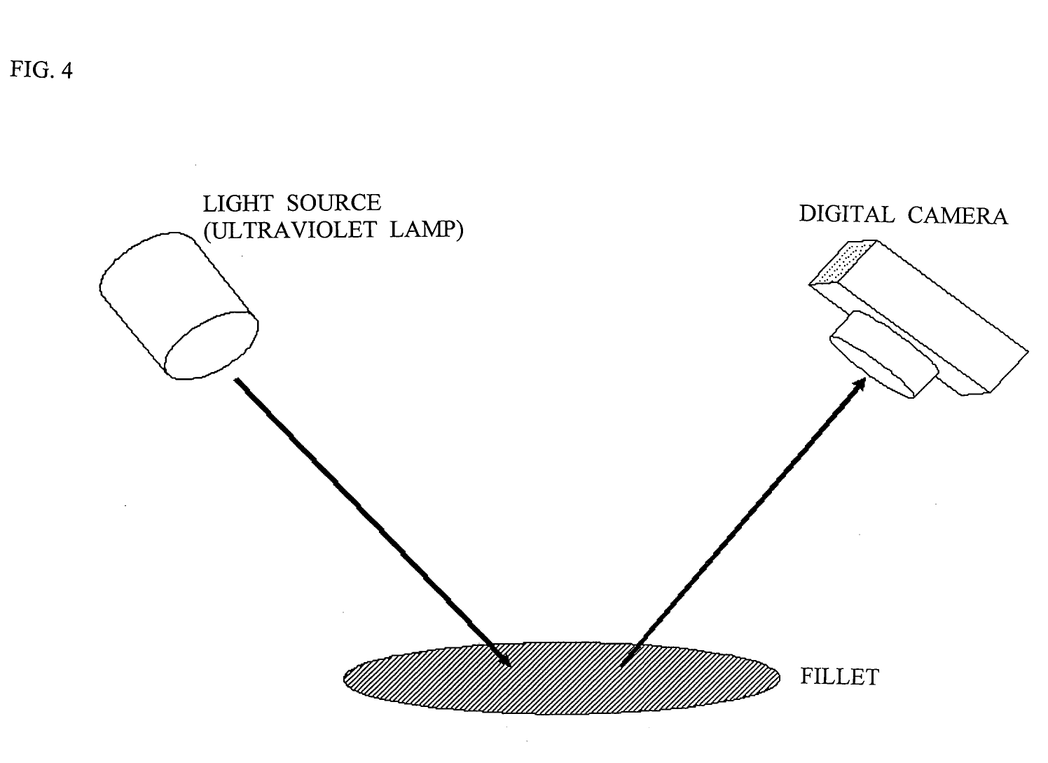

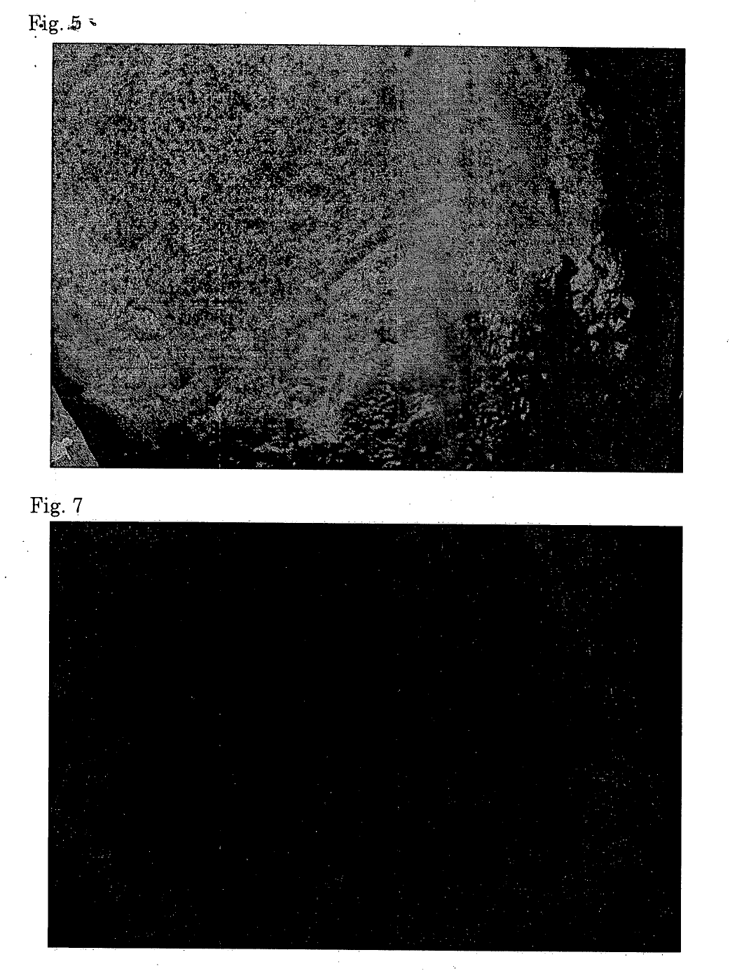

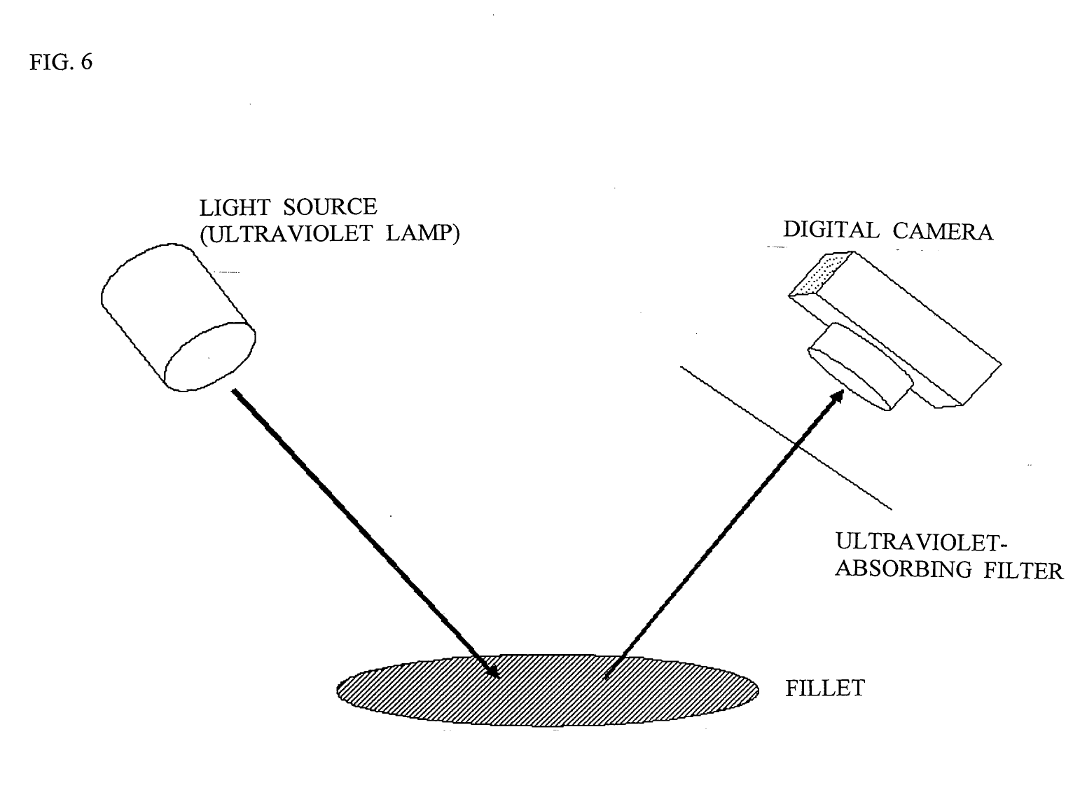

1. A method for detecting a parasite, comprising detecting fluorescence excited by irradiating the parasite with visible light.

2. The method for detecting a parasite according to claim 1, wherein the parasite is parasitic on meat.

3. The method for detecting a parasite according to claim 1, wherein fluorescence emitted from the parasite is selectively detected by using a filter that does not transmit excitation light but that transmits fluorescence emitted from the parasite.

4. The method for detecting a parasite according to claim 1, wherein the parasite is a parasite that fluoresces in response to being subjected to visible-light irradiation.

5. The method for detecting a parasite according to claim 4, wherein the parasite is Ichthyophonus hoferi.

6. A method for detecting foreign matter, comprising detecting fluorescence excited by irradiating meat with visible light.

7. The method for detecting foreign matter according to claim 6, wherein the foreign matter is any of wood, fibers, paper, eggshells, rubber, and insects.

8. The method for detecting a parasite according to claim 2, wherein fluorescence emitted from the parasite is selectively detected by using a filter that does not transmit excitation light but that transmits fluorescence emitted from the parasite.

9. The method for detecting a parasite according to claim 2, wherein the parasite is a parasite that fluoresces in response to being subjected to visible-light irradiation.

10. The method for detecting a parasite according to claim 3, wherein the parasite is a parasite that fluoresces in response to being subjected to visible-light irradiation.

11. The method for detecting a parasite according to claim 8, wherein the parasite is a parasite that fluoresces in response to being subjected to visible-light irradiation.
